(19) United States
(12) Patent Application Publication
(10) Pub. No.: US 2022/0202651 A1
(43) Pub. Date: Jun. 30, 2022

(54) Medicine Delivery Device

(71) Applicant: Lacey Janell Lund, Springdale, AR (US)

(72) Inventor: Lacey Janell Lund, Springdale, AR (US)

(21) Appl. No.: 17/696,440

(22) Filed: Mar. 16, 2022

Related U.S. Application Data
(63) Continuation-in-part of application No. 16/934,217, filed on Jul. 21, 2020, now Pat. No. 11,278,456.

(51) Int. Cl. A61J 7/00; B65D 83/04; A61J 1/14

(52) U.S. Cl. CPC A61J 7/0076; A61J 2200/60; A61J 1/1418; B65D 83/04

(57) ABSTRACT

A medicine delivery device for delivering a predetermined daily dosage of prescription pills to a patient includes a container having a continuous outer wall and a closed bottom that, together, define an open interior area and an open top. Pills, tablets, capsules, and even liquid medicaments may be received in the interior area. A closure member may be releasably coupled to an upper edge of the outer wall for closing the open top of the container. The outer wall of the container consists of a hemp-fiber reinforced biopolymer.

### CROSS-REFERENCE TO RELATED APPLICATIONS

This is a continuation-in-part application that claims the benefit of non-provisional patent application Ser. No. 16/934,217 filed Jul. 21, 2020 and titled Tampon Delivery Device, which is incorporated in its entirety and which was filed by the present inventor and is co-pending. Pursuant to 35 USC 120, 37 CFR 1.78 and MPEP section 211 et seq, the present application repeats a substantial portion of the prior application identified above, and adds disclosure not presented in the prior application. Because this application names the inventor named in the prior application, makes appropriate reference to the earlier application including a priority claim, and is filed before the patenting of the prior application, this continuation-in-part application should be permitted to claim the benefit of the filing date of the earlier non-provisional application.

### FIELD OF THE INVENTION

This invention relates generally to medicine delivery devices and, more particularly, to a pill delivery container having a body portion constructed using a process that includes hemp plastic.

### BACKGROUND OF THE INVENTION

Medications are often ordered by a doctor and dispensed over the counter of a pharmacy and may include a quantity of pills to be ingested by a patient over a number of consecutive days. Accordingly, medications, e.g., pills, tablets, capsules, and the like may be delivered to a patient in what has become known as a “pill bottle” or container such as a traditional “amber” bottle having a lid that is regulated by law to be child-resistant yet openable by an elderly person.

Various patents have been issued for many respective designs that each provide advantages to a consumer's safety as it relates to openability yet flexible enough to be opened by an elderly person. However, the traditional “amber” bottle suffers from another problem that has major consequences both to consumers and especially to the environment. Traditional pill bottles are manufactured using petroleum-based plastics that are filling landfills that are already overfilled with similar oil-based plastics which may take decades if not hundreds of years to decompose.

Therefore, it would be desirable to have a medicine delivery device, also referred to as a pill container, that is strong and robust yet maintains the flexibility needed for use with a pill lid. Further, it would be desirable to have a pill container that is constructed using a material consisting completely or at least partially of hemp plastic so as to apply the benefits therefrom to a patient's health and the environment, and to have a rigid yet flexible construction.

### SUMMARY OF THE INVENTION

The medicine delivery device for delivering a predetermined daily dosage of prescription pills to a patient according to the present invention includes a container having a continuous outer wall and a closed bottom that, together, define an open interior area and an open top. Pills, tablets, capsules, and even liquid medicaments may be received in the interior space. A closure member may be releasably coupled to an upper edge of the outer wall for selectively closing the open top of the container. The outer wall of the container consists of a hemp-fiber reinforced biopolymer.

More particularly, the pill delivery device, is constructed using a material consisting at least partially of hemp plastic so as to apply the benefits therefrom to a patient's health and the environment, and to provide a rigid yet flexible construction. Currently, hemp plastic has not been applied or proposed to patient-care products such as prescription pill containers. Hemp plastic is considered as a sustainable material that has been applied in the past to automobile construction, for concrete, and for building materials due to its strength and anti-denting properties. To date, however, a medicine delivery device such as a pill bottle has not been constructed using hemp materials in that strength and anti-denting properties are not intuitively applied to such devices or seen as feasible. However, the inventor has identified reasons that make it desirable to construct the transparent pill containing and delivery device using forms of hemp plastic. Namely, a cylindrical pill container constructed of a hemp fiber reinforced biopolymer would be biodegradable. This is important for at least two reasons, including that (1) reducing a patient's contact with petrochemical-based plastic is safer, and (2) the pill containing device would be biodegradable and not add to the overburden of landfills that are currently full of traditional plastic products.

Hemp plastic is a bioplastic made using industrial hemp. There are many different types of hemp plastic—from standard plastics reinforced with hemp fibers to a 100% hemp plastic made entirely from the hemp plant. Hemp plastic is recyclable and can be manufactured to be 100% biodegradable.

With even more particular reference to hemp plastic use, hemp plastic is superior over traditional petroleum-based plastic historically used in medicine or pill containers. Specifically, hemp plastic is stronger than petroleum-based products. In fact, plastic made from hemp is five times stiffer and 3.5 times stronger than polypropylene which is one of the most common types of traditional plastic. Hemp plastic is also much lighter than traditional plastic. Being exponentially lighter and stronger make hemp plastic desirable and unique as applied to the present invention. And this makes hemp plastic not only superior—but safer.

Unfortunately, hemp-related products have not been readily available or even pursued with vigor in that cultivation of hemp has been illegal, at least until recently. While illegal inventions are patentable and have indeed received patents, the fact that for decades it has largely been completely illegal to cultivate hemp is evidence of the state of the art, which is relevant to determining obviousness. The U.S. and most countries around the world have, at least until recently, designated marijuana as illegal. See https://www.sternekessler.com/news-insights/publications/cannabis-patenting-all-time-high-despite-illegal-status. Clearly the state of the art regarding hemp-based products is only in its infancy and this can almost certainly be attributed to the fact that Cannabis use has been largely prohibited.

In addition, there has also been little understanding or guidance regarding how to use hemp fiber reinforced biopolymers in industries outside of automobile and construction material industries. The prior art teaches many different types of natural fibers including bamboo, cork, kenaf, hemp, flax, jute, elephant grass, sisal, silk, wool, straw fibers derived from corn, wheat, and rice, pineapple leaf, henequen, Vakka, palm, fibers derived from coir, coconut, and date pits, cotton, wood fiber, abaca, man-made cellulose, seaweed, and avocado. However, the prior art provides no guidance as to why one of skill in the art would select hemp fibers as opposed to any other fiber—and gives no guidance on using a particular fiber to reinforce a biopolymer.

Further, the prior art does not recognize biopolymer composites as being a solution for products such as pill delivery containers. One of the beneficial mechanical properties of fiber-reinforced composites is its strength to weight ratio: the composites can be made very lightweight while still providing high strength. For example, a PLA composite reinforced with Cordenka has an impact strength that is nearly 5× higher than PLA alone. Other fiber-based composites have also exhibited improved strength as compared solely to the biopolymer. Given the positive impact that adding fibers obviously has on the overall strength of a biopolymer composite, it is no surprise that commercial success has been recognized using natural fibers as reinforcements in biopolymers used in automotive and building applications. However, prior art has not heretofore recognized biopolymer composites as a suitable material for medicine dosing devices.

Therefore, a general object of this invention is to provide a medicine delivery device having a pill bottle constructed at least partially of hemp plastic.

Another object of this invention is to provide medicine delivery device, as aforesaid, that is hygienic and safe to the health of patients using the device.

Still another object of this invention is to provide a medicine delivery device, as aforesaid, that is biodegradable.

Yet another object of this invention is to provide a medicine delivery device, as aforesaid, that is stiffer, lighter, and stronger than a medicine delivery device constructed of oil-based plastic yet maintains the necessary flexibility.

A particular object of this invention is to provide a medicine delivery device, as aforesaid, that reinforces a biopolymer using hemp-based fiber.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1a is a perspective view of a medicine delivery device according to a preferred embodiment of the present invention; and

FIG. 1b is an exploded view of the medicine delivery device as in FIG. 1a.

### DESCRIPTION OF THE PREFERRED EMBODIMENT

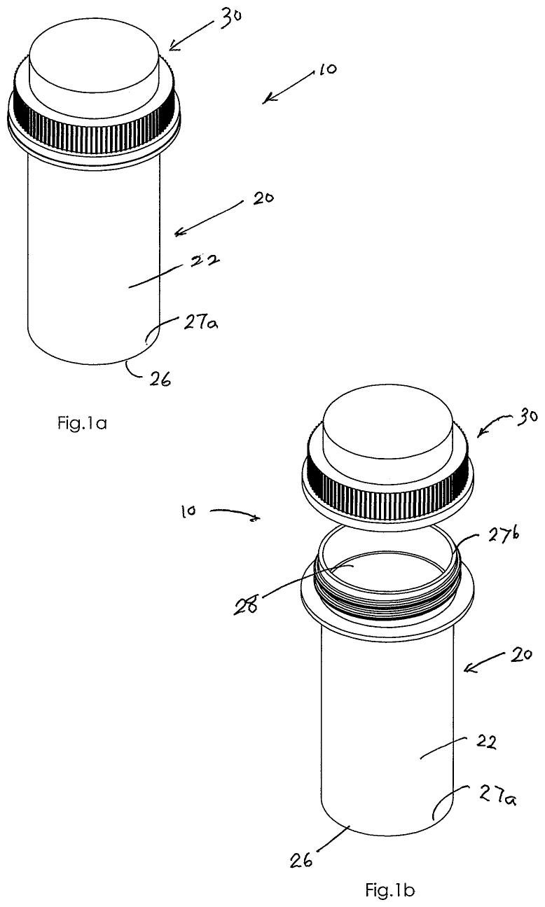

A medicine delivery device according to a preferred embodiment of the present invention will now be described in detail with reference to FIGS. 1a to 1b of the accompanying drawings. The medicine delivery device 10, also referred to as a medicine container or pill container (also using the reference numeral 10), includes a container 20 (a.k.a., a housing) and a closure member 30 (also referred to as a lid 30). Specific reference will be made to a process of making the housing using a process that includes hemp plastic and, specifically, a hemp-fiber reinforced biopolymer (i.e., a composite).

As is described in greater detail herein, in various embodiments of the invention, the housing 20 and lid 30 of the medicine delivery device 10 may be made exclusively, or at least partially, of hemp plastic. No material other than hemp plastic may be necessary or preferred; however, in some embodiments, the addition of biodegradable polymer(s) (a.k.a. biopolymers) may be added to the hemp plastic for construction of the housing. Hemp plastic refers to hemp plant fibers that may be extracted and combined with polymers in a manufacturing process, namely, a hemp-fiber reinforced biopolymer. More particularly, Hemp, or industrial hemp, is a strain of the Cannabis sativa plant species that is grown specifically for the industrial uses of its derived products. A Cannabis fibrous medicine container housing and lid is novel and non-obvious for reasons that are discussed herein.

Hemp plastic can be entirely biodegradable when made or combined with biodegradable polymers. A biodegradable polymer is a polymer that can be decomposed by bacteria. Examples of biodegradable polymers which can broken down by micro-organisms within a suitable amount of time include polyglycolic acid (PGA), polyhydroxy butyrate (PHB), polyhydroxy butyrate-co-beta hydroxyl valerate (PHBV), polycaprolactone (pcl), nylon-2-nylon-6. If made properly and in the right environment, hemp plastic can decompose in 3 to 6 months which is unexpected in view of petroleum-based plastic which are toxic and degrade very little even after decades. Hemp plastic may also be reinforced with fibers.

Now with further reference to the medicine delivery device 10, the medicine delivery device 10 may include a container 22 having a continuous outer wall 24 coupled to a closed bottom 26 that, together with the continuous outer wall 24, defines an interior area for receiving and securing medicine such as pills, tablets, capsules, and even liquids such as cough syrup or the like. More particularly, the continuous outer wall 24 may include a lower edge 27a connected to the bottom 26 in a unitary construction or sealed construction. Further, the continuous outer wall 24 may include an upper edge 27b opposite the lower edge 27a and which defines an open top 28. The upper edge 27b may be threaded for receiving the closure member 30. Specifically, the closure member 30 may be a circular lid (also referred to with reference 30) that is threaded and releasably coupled to the threaded upper edge 27b of the container 20.

Preferably, the continuous outer wall 24 of the container 20 has a cylindrical configuration typical of a traditional pill bottle; however, the container 20 may have other configurations such as that of a rectangular, circular, or the like.

Now with further reference to the construction of the medicine delivery device 10, the container 20 and closure member 30, may consist essentially of hemp plastic rather than petrochemical plastics. In the embodiments, the basic and novel characteristic of the invention is that the container 20 are constructed of hemp plastic such that the medicine delivery device is entirely biodegradable and is at least two times stronger than conventional applicators while maintaining the flexibility necessary to operate the applicator according to standard operation. It is understood that the following discussion regarding construction/manufacture may refer to specific elements of the container 20, such as reference to the continuous outer wall 24, bottom 26, or closure member 30.

In some embodiments, the material forming the medicine delivery device 10, and more specifically, the container 20 and the closure member 30, consists essentially of a combination of a hemp plastic and a biodegradable polymer or polymers. Here, the basic and novel characteristics of the invention are that the applicator is formed essentially of a combination of hemp plastic and a biodegradable polymer such that the applicator is biodegradable and maintains a strength of at least two times the strength of conventional plastics.

More particularly, hemp plants may be harvested, broken down into essential components for production of the container 20 and closure member 30 of the medicine delivery device 10. Constructed of hemp plastic, the continuous outer wall 22 and, in an embodiment where both the continuous outer wall 22 and lid 30 are constructed of hemp plastic, the overall pill container 10 exhibits a firmness and certain lack of flexibility that is both unexpected and highly desirable, while maintaining the necessary flexibility such that operation of the closure member 30 may proceed according to conventional wisdom. Specifically, a medicine delivery device 10 constructed of hemp-plastic overcomes problems typical with pill containers constructed of traditional or petroleum-based plastic. The advantages of hemp plastic are only experienced when the medicine delivery device 10 recited in the present claims is constructed essentially of hemp plastic, or a combination of hemp plastic and biodegradable polymer(s).

According to further embodiments, the medicine delivery device 10 may be constructed only of hemp plastic, with only slight amounts of naturally occurring impurities or impurities that are introduced by virtue of the processing process, or include a hemp-fiber reinforcement to the biopolymer (i.e., a composite). In such embodiments, the applicator may exhibit strength properties that are superior to conventional pill containers.

In still other embodiments, the medicine delivery device 10 is constructed only of hemp plastic together with a biodegradable polymer, such as the biodegradable polymers described herein. In such embodiments, the medicine delivery device 10 may further include slight amounts of naturally occurring impurities, or impurities that are introduced into the material during the manufacturing process. According to some aspects, the medicine delivery device 10 is constructed of about 1-100 wt % hemp plastic and about 0-99 wt % biodegradable polymer or polymers. In further aspects, the medicine delivery device 10 is constructed of about 10-100 wt %, about 20-100 wt %, about 30-100 wt %, or about 40-100 wt % hemp plastic, and the remainder biodegradable polymer or polymers. According to still further aspects, the medicine delivery device 10 consists of at least about 50 wt % hemp plastic and the remainder biodegradable polymer or polymers. In some embodiments it may be preferable for the amount of hemp plastic to be greater than about 50 wt %, greater than about 60 wt %, greater than about 70 wt %, greater than about 80 wt %, or greater than about 90 wt %, with the remainder, if any, being biodegradable polymer or polymers.

In use, a pharmacist inserts the medicine into the interior area through the open top 28 defined by the continuous outer wall 22 and closed bottom 26. A patient, may then remove pills from the interior area according to the dosing indicia that may be imprinted on a label wrapped around the continuous outer wall 24.

It is understood that while certain forms of this invention have been illustrated and described, it is not limited thereto except insofar as such limitations are included in the following claims and allowable functional equivalents thereof.

1. A medicine delivery device for delivering a predetermined daily dosage of prescription pills, comprising:
a container having a continuous outer wall and a closed bottom that, together, define an open interior area and an open top;
a closure member for selectively closing the open top of the container;
wherein said container consists of a hemp-fiber reinforced biopolymer.

2. The medicine delivery device as in claim 1, wherein said container is biodegradable.

3. The medicine delivery device as in claim 2, wherein said container consists only of hemp plastic material.

4. The medicine delivery device as in claim 1, wherein said container is a pill bottle and said continuous outer wall has a cylindrical configuration.

5. The medicine delivery device as in claim 4, wherein said container consists of hemp-plastic and optionally a biodegradable polymer.

6. The medicine delivery device as in claim 1, wherein said closure member consists of a hemp-fiber reinforced biopolymer.

7. A medicine delivery device for delivering a predetermined daily dosage of prescription pills, comprising:
a container having a continuous outer wall and a closed bottom that, together, define an open interior area and an open top;
a closure member for closing the open top of the container;
wherein said continuous outer wall and said closure member are comprised of about 1-100 wt % of hemp plastic material.

8. The medicine delivery device as in claim 7, wherein said continuous outer wall is biodegradable.

9. The medicine delivery device as in claim 8, wherein said continuous outer wall further comprises about 0-99 wt % of at least one biodegradable polymer.

10. The medicine delivery device as in claim 9, wherein said closure member is completely biodegradable.

11. The medicine delivery device as in claim 10, wherein said closure member further comprises about 0-99 wt % of at least one biodegradable polymer.

12. A medicament container for securing a predetermined quantity of prescription pills, comprising:
a container including a continuous outer wall having a cylindrical and transparent configuration constructed of hemp plastic material that includes at least one biodegradable polymer and a plurality of hemp fibers, said continuous outer wall having a lower edge coupled to a closed bottom that, together, define an open interior area and an open top through which said predetermined quantity of prescription pills are received into said open interior area; and
a closure cap consisting of hemp plastic material comprising at least one biodegradable polymer and a plurality of hemp fibers, said closure cap being threadably coupled to an upper edge of said continuous outer wall for selectively allowing and disallowing access to said open interior area via said open top.

13. The medicine delivery device as in claim 14, wherein said one biodegradable polymer of said continuous outer wall is the same as said at least one biodegradable polymer of said closure cap.
